(19) United States
(12) Patent Application Publication
(10) Pub. No.: US 2018/0003701 A1
(43) Pub. Date: Jan. 4, 2018

(54) IMMUNOGLOBULIN ASSAYS USING NANOPARTICLES

(71) Applicant: AGENCY FOR SCIENCE. TECHOLOGY AND RESEARCH, Singaporesg (SG)

(72) Inventors: Laura SUTARLIE, Singapore (SG); Yan Nee TAN, Singapore (SG); Yong YU, Singapore (SG); Xiaodi SU, Singapore (SG); Dave Siak-Wei OW, Singapore (SG); Say Kong NG, Singapore (SG); Yuan Sheng YANG, Singapore (SG)

(21) Appl. No.: 15/545,674

(22) PCT Filed: Jan. 21, 2016
(86) PCT No.: PCT/SG2016/050025

§ 371 Date: Jul. 21, 2017

(30) Foreign Application Priority Data
Jan. 21, 2015 (SG) 10201500166S

(51) Int. Cl. G01N 33/542; G01N 33/543; G01N 33/533; G01N 33/58; G01N 33/53

(52) U.S. Cl. CPC G01N 33/542; G01N 33/54346; G01N 33/533; G01N 33/54393; G01N 33/5302; G01N 33/587

(57) ABSTRACT

A system for measuring presence and/or amount of a target antibody in a sample comprising a nanoparticle adapted to quench fluorescence emission, an Fc binding protein immobilized or absorbed on the nanoparticle, and a fluorescing antibody adapted for binding to the Fc binding protein and having a lower binding affinity to the Fc binding protein than the target antibody. According to further embodiments of the present invention, methods of measuring presence and/or amount of a target antibody in a sample are also provided. The methods comprise mixing the sample with the components of the system and measuring the change in fluorescent intensity as compared to the total fluorescent intensity before the mixing. The lower affinity of the fluorescing antibody to the Fc binding protein as compared to the target antibody allows for the fluorescing antibody to be replaced by the target antibody and stay unbound in the solution, causing a change in the fluorescence quenching, thereby enabling an estimate on the amount of the target antibody in the sample.

### CROSS-REFERENCE TO RELATED APPLICATIONS

This application claims the benefit of priority of Singapore patent application No. 10201500466S, filed 21 Jan. 2015, the contents of it being hereby incorporated by reference in its entirety for all purposes.

### FIELD OF THE INVENTION

The present invention relates to biochemistry in particular preparation of proteins for use as capturing agents. In particular, the present invention relates to systems for measuring presence and/or quantifying the amount of a target antibody in a sample.

### BACKGROUND OF THE INVENTION

Antibodies as biological drugs for human in biopharmaceuticals are manufactured using mammalian cell lines, where the antibody titer needs to be measured and quantified for cell line selection and screening. Whilst methods for detecting antibodies are known in the art, there is still a need to provide an alternative method for measuring and detecting antibodies in samples in a faster manner.

Accordingly, there is a need to provide an alternative method for detecting antibodies in a sample in a faster manner.

### SUMMARY OF THE INVENTION

In one aspect, there is provided a system for measuring presence and/or amount of a target antibody in a sample. The system comprises a nanoparticle capable of or adapted to quenching fluorescence emission. The system further comprises an Fc binding protein, wherein the Fc binding protein is immobilized or absorbed on the nanoparticle. The system also comprises a fluorescing antibody capable or adapted for binding to the Fc binding protein and having a lower binding affinity to the Fc binding protein than the target antibody.

In another aspect, there is provided a method of measuring presence and/or amount of a target antibody in a sample. The method comprises mixing components of a system as described herein and the sample. The method further comprises the step of measuring the change in fluorescent intensity observed in the mixture obtained in the first step as compared to the total fluorescent intensity of the fluorescing antibody before the mixing step in the first step, wherein a decrease in fluorescent intensity indicates the presence and/or amount of the target antibody in the sample in an inverse relationship.

In yet another aspect, there is provided a second method of measuring presence and/or amount of a target antibody in a sample. In one example, the method comprises mixing the sample with the components of the system as described herein. The method further comprises the step of measuring the change in fluorescent intensity observed in the mixture obtained in the first step as compared to the total fluorescent intensity of the system before the mixing in the first step, wherein an increase in fluorescent intensity indicates the presence of target antibody in the sample.

### BRIEF DESCRIPTION OF THE DRAWINGS

The invention will be better understood with reference to the detailed description when considered in conjunction with the non-limiting examples and the accompanying drawings, in which:

FIG. 1A) shows an illustration of the structure of an immunoglobulin G (i.e. IgG). Immunoglobulin G (IgG) is composed of four peptide chains, which includes two identical heavy chains and two identical light chains arranged in a Y-shape typical of antibody monomers. Each IgG has two antigen-binding sites (termed Fab regions), which bind to the epitope of the target antigen, and one constant region (Fc region), which interact with cell surface receptors called Fc receptors and some proteins of the complement systems; B) shows an illustration of the structure of an example of Fc-binding proteins immobilized on a metal nanoparticle.

FIG. 2 shows a schematic illustration of an example of a displacement assay as described herein. In this example, the displacement assay is for human IgG. In one example, the assay component is fluorescent quenched rat IgG* (i.e. fluorescent-labeled rat IgG which fluorescent label has been quenched/dampened) bound on protein G-gold nanoparticle (pG-AuNPs). The component is referred herein as rat IgG*-pG-AuNPs. Human IgG is quantified based on fluorescence intensity increase due to the displacement of rat IgG* by human IgG. In the right panel, A and B represent organic dye-labeled or intrinsic fluorescent rat IgG*, respectively. In one example, the components of FIG. 2 may be replaced with equivalent components known in the art and as described herein. For example, the assay component may be fluorescent quenched antibody* (i.e. fluorescent-labeled antibody which fluorescent label has been quenched/dampened) bound on Fc-binding protein-nanoparticle. Target antibody may then be quantified based on fluorescence intensity increase due to the displacement of antibody* by target antibody. In the right panel, A and B represent organic dye-labeled or intrinsic fluorescing antibody*, respectively. The incubation period may be between 10 minutes to 50 minutes.

FIG. 3 shows dot-plots showing the fluorescence intensity increase upon displacement of rat IgG labeled with an Alexa Fluoro 488 (A488) fluorophore (i.e. fluorescent dye) by monoclonal antibodies, Herceptin, Avastin, and Humira at varying concentrations (about 10 to 1000 mg/L). In particular, FIG. 3 shows percentage of fluorescence increase as a function of the concentration of (a) Herceptin in phosphate buffered saline (PBS), (b) Herceptin in cell supernatant, (c) Avastin in cell supernatant, and (d) Humira in cell supernatant by using one example of the system as described herein (e.g. displacement assay). FIG. 3(a) to (d) shows all of the antibodies tested resulted in increase of fluorescence intensity. FIG. 3(e) shows a graph depicting the changes of fluorescence spectrum of rat IgG* in the displacement assay. The original fluorescence spectrum of free rat IgG* (dashed line) is quenched to F₀ (dotted line) when the rat IgG* are bound by pG-AuNPs into the composite of rat IgG*-pG-AuNPs. The fluorescence recovers from F₀ to Fₛ (solid line) after human IgG analyte addition as the human IgG analyte displaces rat IgG* from the composite. F₀ to Fₛ are as described in FIG. 2 above. Thus, FIG. 3 shows one example of the system as described herein (i.e. displacement assay) can be used to quantify various therapeutic human IgG.

FIG. 4 shows graphs plotting the fluorescence intensity increase upon displacement of rat IgG labeled with AuNCs by a monoclonal antibody, Herceptin, at varying concentration of between 2.7 to 2700 mg/L. In particular, a) shows a graph of the photoemission spectra (λex=370 nm) of rat IgG-Au nano-composite/complex (control, dashed line), the composite/complex of rat IgG-Au nano-composite/complex mixed with pG-coated with 13 nm gold nanoparticles (AuNPs; dotted line), and mixture of rat IgG-Au nano-composite, pG-coated gold nanoparticle (AuNPs) and Herceptin (2.7 g/L, 5 μL; solid line); b) shows a bar-graph of the normalised fluorescence intensity to demonstrate the antibody assay based on the displacement principle; c) shows a dot-plot of the percentage fluorescence increase as a function of Herceptin concentration in the sample dissolved in phosphate buffered saline (PBS) using a displacement assay. Thus, FIG. 4 shows the linear response correlated to the concentration of sample IgG detected using an example of the displacement assay as described herein.

FIG. 5 shows a dot-plot showing the percentage of fluorescence increase as a function of goat anti-biotin concentration in phosphate buffered saline (PBS) by using displacement assay. FIG. 5 shows that the goat anti-biotin tested resulted in fluorescence intensity increase upon displacement of rat IgG labeled with an Alexa Fluoro 488 (A488) fluorophore (i.e. fluorescent dye) by goat anti-biotin.

FIG. 6 shows a schematic illustration of an example of a competition assay as described herein. In this example, the competition assay is for human IgG. In one example, the assay has two components of: a) Assay component 1, which is composed of rat IgG* (i.e. fluorescing rat IgG); and b) Assay component 2, which is composed of protein G-gold nanoparticles (AuNPs). In one example, the assay involves two steps of sample mixing, namely 1) target sample with assay component 1 (i.e. IgG analyte/target with rat IgG*), followed by 2) incubation with assay component 2 (i.e. pG-AuNPs) conjugates for 15 mins.

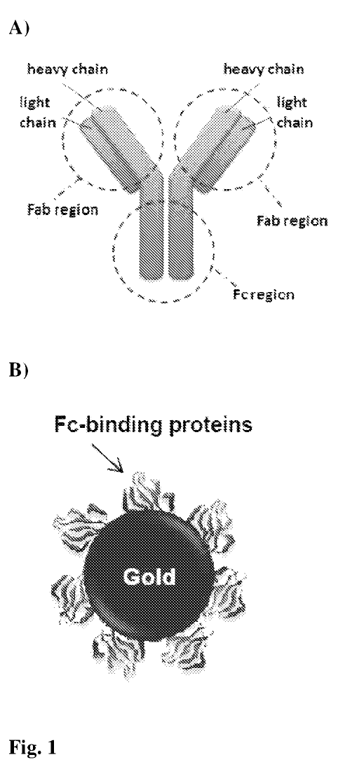

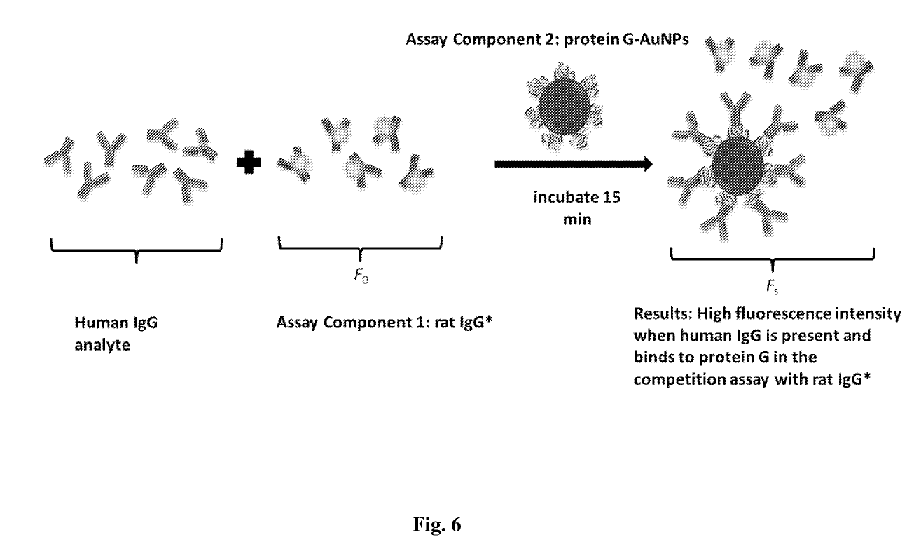

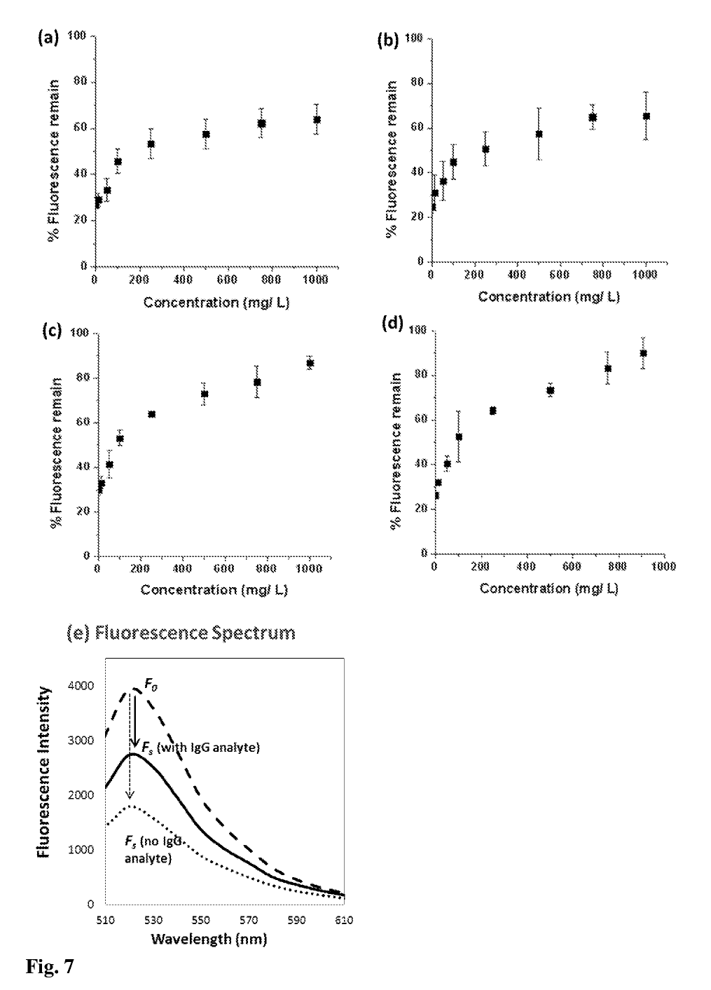

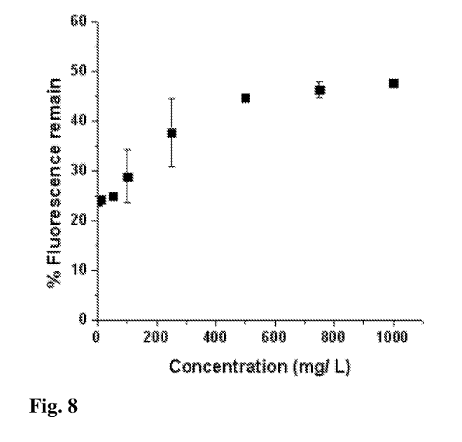

In one example, the components of FIG. 6 may be replaced with equivalent components known in the art and as described herein. For example, the assay component may have two components of: a) Assay component 1, which is composed of fluorescing antibody*; and b) Assay component 2, which is composed of Fc-binding protein-nanoparticles. In one example, the assay involves two steps of sample mixing, namely 1) target antibody with fluorescing antibody* of assay component 1, followed by 2) incubation with Assay component 2 (Fc-binding protein nanoparticle conjugates) for suitable incubation period, such as from about 10 minutes to 50 minutes.

FIG. 7 shows dot-plots of the remaining fluorescence intensity in competition assay samples containing monoclonal antibodies Herceptin, Avastin, and Humira at varying concentrations of between 10 to 1000 mg/L. In particular, FIG. 7 shows the remaining fluorescence intensity in competition assay as a function of the concentration of (a) Herceptin in phosphate buffered saline (PBS), (b) Herceptin in cell supernatant, (c) Avastin in cell supernatant, (d) Humira in cell supernatant, (e) shows a graph depicting the change of fluorescence of F₀ (the original fluorescence spectrum of free rat IgG*, dashed line) to Fₛ with IgG analyte (solid line) or Fₛ no IgG analyte (dotted line) after addition of pG-AuNPs in the competition assay. If there is no IgG analyte, all rat IgG* are bound to pG-AuNPs, and the fluorescence is quenched. In the presence of IgG analyte, IgG analyte competes with rat IgG* to bind to pG, leaving some free rat IgG* remain fluorescent. F₀ and Fₛ are indicated in FIG. 6 above. Thus, FIG. 7 shows the system as described herein can be used in a competition assay to detect antibodies.

FIG. 8 shows a dot plot of the percentage of remaining fluorescence in competition assay as a function of goat anti-biotin antibody concentration in PBS. FIG. 8 shows the applicability of competition assay for quantifying IgG from other sources.

### DETAILED DESCRIPTION OF THE PRESENT INVENTION

Methods for detecting the presence and/or amount of an agent in a sample are in great demand across fields of science, medicine, agriculture and the like. Whilst such methods are widely available, there is still a need to provide alternative methods for detecting the presence and/or amount of an agent in a sample.

The inventors of the present disclosure found a cost effective and rapid method of detecting the presence and/or amount of an agent in a sample. In one example, the present disclosure describes the methods for detecting and measuring titer of antibodies (such as monoclonal antibodies) that exploits the fluorescence quenching properties of metal nanoparticles. In principle, metal nanoparticles with small diameter are found to be super quencher of proximate fluorophore (within tens of nm) through fluorescence resonance energy transfer (FRET) or surface electron transfer. By using this principle, the inventors of the present disclosure developed methods for detecting the presence and/or amount of an agent in a sample that is convenient, rapid and low cost.

Thus, in one aspect, there is provided a system for measuring presence and/or amount (titer) of a target antibody in a sample. In one example, the system may comprise or consists of a nanoparticle capable of or adapted to quenching fluorescence emission; an Fc binding protein, wherein the Fc binding protein is immobilized or absorbed on the nanoparticle; and a fluorescing antibody capable or adapted for binding to the Fc binding protein and having a lower binding affinity to the Fc binding protein than the target antibody.

As used herein, the term “nanoparticle” refers to a composite structure of nanoscale dimensions. In one example, the nanoparticle may be particles of a size in the range of from about 1 to 1000 nm, and may be uniform or non-uniform in structure. In one example, the nanoparticle may have various morphologies that are possible depending on the nanoparticle composition. In one example, the nanoparticles as described herein may be isotropic in dimension. That is, the nanoparticles as used herein may be uniform in shape or having a physical property that has the same value when measured in different directions. Thus, in one example, the nanoparticles as described herein may be spherical nanoparticles. In one example, the nanoparticles may be a rod nanoparticle. For example, the rod nanoparticle may be nanorod. In another example, the nanoparticles as described herein may be anisotropic in dimension. That is, the nanoparticles as used herein may be different in shape or have a physical property that has a different value when measured in different directions. In one example, the anisotropic nanoparticles may include, but is not limited to, nanoflowers, nanostars, and the like.

As would be understood by the person skilled in the art, the desired properties of a nanoparticle may depend on the material and size of the nanoparticle. In one example, the nanoparticle may have desirable properties of nanoparticles, such as surface charges, steric stabilization, and/or plasmonics fluorescence quenching capabilities/properties. In one example, the nanoparticle may have fluorescence quenching properties. In one example, the nanoparticle may have plasmonics fluorescence quenching properties. In one example, metal nanoparticles with small diameter, such as a diameter of less than 80 nm are found to be super quencher of proximate fluorophore (within tens of nm) through fluorescence resonance energy transfer (FRET) or surface electron transfer. Thus, in one example, the nanoparticle having fluorescence quenching properties may have suitable size for quenching the fluorescence of a fluorophore bound to an antibody and/or the fluorescence of an intrinsically fluorescing antibody. In one example, the nanoparticle may have a diameter from about 2 nm to about 80 nm in diameter, or from about 3 nm to about 70 nm in diameter, or from about 5 nm to about 50 nm, or from about 6 nm to 20 nm. In one example, the nanoparticle may have a diameter of less than about 80 nm, or less than about 75 nm, or less than about 70 nm, or less than about 65 nm, or less than about 60 nm, or less than about 55 nm, or less than about 50 nm, or less than about 45 nm, or less than about 40 nm, or less than about 35 nm, or less than about 30 nm, or less than about 25 nm, or less than about 20 nm, or less than about 19 nm, or less than about 18 nm, or less than about 17 nm, or less than about 16 nm, or less than about 15 nm, or less than about 14 nm, or less than about 13 nm, or less than about 12 nm, or less than about 11 nm, or less than about 10 nm. In one example, the nanoparticle may have a diameter of about 13 nm, or about 50 nm. As used herein, the term “about”, in the context of diameter of a nanoparticle, may refer to +/−5% of the stated value, or +/−4% of the stated value, or +/−3% of the stated value, or +/−2% of the stated value, or +/−1% of the stated value, or +/−0.5% of the stated value.

As used herein, the term “plasmonic nanoparticles” refers to nanoparticles that have very strong absorption (and scattering) spectrum that is determined by the shape, the composition or the medium around their surfaces. It will be appreciated that the term includes all plasmonic nanoparticles of various shapes and surface surrounding which gives them surface plasmon absorption and scattering spectrum in the visible-near infra-red region of the spectrum.

In one example, the nanoparticle may be a metal nanoparticle. In one example, the nanoparticle may be a metal nanoparticle with plasmonics fluorescence quenching property. In one example, the nanoparticle may be a noble metal nanoparticle including, but is not limited to, ruthenium, rhodium, palladium, silver, osmium, iridium, platinum, gold, and the like, as well as copper nanoparticle, and core-shell nanoparticles. In one example, the nanoparticle may be a gold nanoparticle (AuNP), a silver nanoparticle, or a copper nanoparticle. In one example, the nanoparticle may be a gold nanoparticle (AuNP).

As used herein, the term “quenching” refers to a reduction in the luminescent intensity of the luminescent material. In one example, the luminescent material, as described herein, is a material that emits light when activated or in response to radiant energy. In one example, the luminescent is fluorescence. Thus, in the context of the present disclosure, the quenching of fluorescence refers to the sequestering or reduction of the fluorescence of a fluorescing material. In one example, the quenching of fluorescence may occur when the fluorescing material is in close proximity with a quenching material. In one example, the quenching of fluorescence emission may occur when the nanoparticle absorption spectra overlaps with the emission spectrum of fluorescence to be absorbed.

In one example, the degree of quenching may be evaluated qualitatively, by comparing the luminescence intensity of a sample with an emitter and a test quenching material to the luminescence intensity of a control sample. The control sample can be the emitter without any quenching material, or the emitter with a charge transport and/or quenching material known to have a low quenching constant. The qualitative method may be used to screen large numbers of materials using combinatorial techniques.

In one example, the luminescence may be fluorescence which may have an emission peak of between about 300 nm to about 800 nm, or between about 350 nm to about 700 nm, or between about 400 to about 670 nm. Thus, in one example, the fluorescence may be emitted by fluorophores such as, but are not limited to, Cy 3, Cy 3B, organic dye like Alexa Fluoro 405, Alexa Fluor 430, Alexa Fluor 488, Alexa Fluor 546, Alexa Fluor 610-R-phycoetrythrin (R-PE), Alexa Fluor 633, Alexa Fluor 635, Alexa Fluor 647, Alexa Fluor 647-R-phycoetrythrin (R-PE), Alexa Fluor 660, Alexa Fluor 680, Alexa Fluor 680-allophycocyanin, Alexa Fluor 680-R-phycoerythrin, Alexa Fluor 700, Alexa Fluor 750, Alexa Fluor 750-Allophycocyanin (APC), APC (Allophycocyanin), Cascade Blue, Cascade Yellow, Cy5.5-Allophycocyanin (APC), Fluorescein (FITC), Marina Blue, Oregon Green 488, Pacific Blue, Pacific Green, Pacific Orange, PerCP-Cy5.5, pHrodo Red, Qdot 525, Qdot 605, Qdot 625, Qdot 655, Qdot705, Qdot 800, R-PE (R-phycoerythrin), R-PE (R-phycoerythrin)-Alexa Fluor 700, R-PE (R-phycoerythrin)-Alexa Fluor 700, R-PE (R-phycoerythrin)-Cy7, R-Phycoerythrin (R-PE)-Cy5.5, Texas Red-R-Phycoerythrin (R-PE), blue TRI-COLOR, green Tri-COLOR, yellow TRI-COLOR, orange TRI-COLOR, and the like.

In one example, when the quencher is a nanoparticle, the nanoparticle is a gold nanoparticle. In one example, when the quencher is a gold nanoparticle, the fluorophores may include, but are not limited to, Cy 3, Cy 3B, organic dye like Alexa Fluoro 405, Alexa Fluor 430, Alexa Fluor 488, Alexa Fluor 546, Alexa Fluor 610-R-phycoetrythrin (R-PE), Alexa Fluor 633, Alexa Fluor 635, Alexa Fluor 647, Alexa Fluor 647-R-phycoetrythrin (R-PE), APC (Allophycocyanin), Cascade Blue, Cascade Yellow, Fluorescein (FITC), Marina Blue, Oregon Green 488, Pacific Blue, Pacific Green, Pacific Orange, PerCP-Cy5.5, pHrodo Red, Qdot 525, Qdot 605, Qdot 625, Qdot 655, R-PE (R-phycoerythrin), Texas Red-R-Phycoerythrin (R-PE), and the like. In one example, when the diameter of the gold nanoparticle is less than 13 nm, the fluorophores may include, but are not limited to, Cy 3, Cy 3B, organic dye like Alexa Fluoro 405, Alexa Fluor 430, Alexa Fluor 488, Alexa Fluor 546, Alexa Fluor 610-R-phycoetrythrin (R-PE), Alexa Fluor 633, Alexa Fluor 635, Alexa Fluor 647, Alexa Fluor 647-R-phycoetrythrin (R-PE), APC (Allophycocyanin), Cascade Blue, Cascade Yellow, Fluorescein (FITC), Marina Blue, Oregon Green 488, Pacific Blue, Pacific Green, Pacific Orange, PerCP-Cy5.5, pHrodo Red, Qdot 525, Qdot 605, Qdot 625, Qdot 655, R-PE (R-phycoerythrin), Texas Red-R-Phycoerythrin (R-PE), and the like.

The term “immobilized” or “adsorbed”, as used herein, is intended to encompass direct or indirect, covalent or non-covalent attachment, unless indicated otherwise, either explicitly or by context. In one example, the Fc binding protein may be immobilized or absorbed to the nanoparticle by covalent binding. In one example, the Fc binding protein may be immobilized to the nanoparticle by covalent binding of the Fc binding protein with the nanoparticle through cysteine residues within the Fc binding protein. In one example, the Fc binding protein may be immobilized to the nanoparticle by covalent binding of the Fc binding protein with the gold nanoparticle through cysteine residues within the Fc binding protein.

As used herein, the “Fc binding protein” refers to proteins that can bind to the Fc region of an antibody. In one example, the Fc binding protein is not an immunoglobulin that recognizes immunoglobulin (such as IgG) through antigen binding site. In one example, the Fc binding protein is a non-immune protein. In one example, the Fc binding protein may include, but is not limited to, Fc receptors (such as Fc receptors found in certain cells), protein G (pG) (such as when the antibody is IgG), protein A (pA) (such as when the antibody is IgG), and fusion protein G/A (pG/A) (such as when the antibody is IgG).

Additionally, without wishing to be bound by theory, in one example, the Fc binding protein of the present disclosure may be adapted to or may be capable of differentially binding to the Fc region of the target antibody such that the target antibody from different animals can be detected (for example, as demonstrated for human IgG and goat IgG) relative to other Ig classes (such as IgA and IgM)). Accordingly, in one example, the Fc binding protein may be adapted to or capable of differentially binding to the fluorescing antibody and the target antibody. The phrase “differentially binding” refers to the selective binding of the non-immune protein Fc binding protein to the Fc region of the target antibody.

The term “antibody” is used herein in the broadest sense and refers to an immunoglobulin or fragment thereof, and encompasses any such polypeptide comprising an antigen-binding fragment or region of an antibody. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Immunoglobulin classes may also be further classified into subclasses, including IgG subclasses IgG₁, IgG₂, IgG₃, and IgG₄; and IgA, subclasses IgA₁ and IgA₂. The term antibody includes, but is not limited to, polyclonal, monoclonal, monospecific, multispecific (for example, bispecific antibodies), natural, humanized, human, chimeric, synthetic, recombinant, hybrid, mutated, grafted, antibody fragments (e.g., a portion of a full-length antibody, generally the antigen binding or variable region thereof, e.g., Fab, Fab′, F(ab′)₂, and Fv fragments), and in vitro generated antibodies so long as they exhibit the desired biological activity. The term also includes single chain antibodies, e.g., single chain Fv (sFv or scFv) antibodies, in which a variable heavy and a variable light chain are joined together (directly or through a peptide linker) to form a continuous polypeptide. In one example, the term “antibody”, as used herein, may be an immunoglobulin G (IgG) selected from the group consisting of Immunoglobulin G (IgG), Immunoglobulin A (IgA), Immunoglobulin M (IgM), Immunoglobulin E (IgE) and Immunoglobulin D (IgD).

As used herein, the term “antibody” refers to both the target antibody (i.e. the antibody to be detected in the sample) and the fluorescing antibody (i.e. one of the components of the system as described herein). The antibody that forms part of the components of the system as described herein is a fluorescing antibody. As used herein, the phrase “fluorescing antibody” may refer to either an antibody that intrinsically fluoresced or an antibody that have been labelled with a fluorophore (i.e. organic dye and quantum dots (QDs)). In one example, the antibody, which intrinsically fluoresced, may be an antibody template nanocluster. In another example, the antibody may be a template metal nanocluster.

For example, the antibody may be a template gold nanocluster. In one example, the intrinsically fluorescing antibody may be an immunoglobulin template nanocluster, an immunoglobulin template metal nanocluster, an immunoglobulin template gold nanocluster, or gold nanocluster that is synthesized using an antibody as a template. In one example, the antibody template nanocluster or antibody template gold nanocluster may be prepared by mixing the antibody, a nanoparticle precursor solution and a suitable basic solution as described herein. In one example, the antibody template (gold) nanocluster is prepared by mixing the antibody, a nanoparticle precursor solution (such as HAuC1₄ solution) and a suitable basic solution (such as NaOH).

Without wishing to be bound by theory, the systems as described herein rely on differential binding affinity of two immunoglobulins (such as IgGs) to Fc binding proteins conjugated on nanoparticles (such as gold nanoparticles). One of the immunoglobulin (Ig) is the target or analyte immunoglobulin (i.e. Ig target/analyte, Ig to be quantified), and the other immunoglobulin (Ig to be displaced or competing Ig), which is a fluorescent Ig from a different animal source that has a lower affinity to the Fc binding protein than the Ig target/analyte (i.e. Ig to be detected/quantified). Thus, in one example, the antibody that forms part of the component of the system as described herein may be an immunoglobulin that has a relatively low affinity to the selected Fc binding protein relative to the target immunoglobulin (i.e. analyte Ig). The lower affinity of the antibody of the system as compared to the target/analyte antibody allows for the antibody of the system to be replaced or removed by the presence of the target/analyte antibody. The replacement or removal of the antibody that forms part of the component of the system by the target/analyte antibody results in the change of overall fluorescence detected in the sample. Further detail on how the change in fluorescence can be used to detect/measure antibody is described in more detail further below. In one example, the antibody may be an immunoglobulin G (IgG) having a relatively low affinity to the selected Fc binding protein relative to the target (analyte) IgG. In one example, the antibody may be an immunoglobulin G (IgG) having a relatively low affinity to the selected Fc binding protein relative to the target (analyte) IgG. For example, the antibody may be a rat immunoglobulin G (r IgG) having a relatively low affinity to the selected Fc binding protein relative to the target (analyte) human IgG (h IgG). When designing the system as described herein, the person skilled in the art would have to determine the suitability of the immunoglobulin of the system with respect of the immunoglobulin to be detected. To determine the binding affinity, common general knowledge in the art, such as that described in Thermo Scientific, Binding characteristics of Antibody-binding Proteins, 2013, may be consulted.

For example, in one example, if the target/analyte is human IgG, the Fc binding protein may be protein G (pG) that is conjugated to a nanoparticle (such as AuNPs) and a fluorescent IgG from rat (see Examples 1 and 2, where the fluorescing IgG from rat is denoted as rat IgG*). In another example, human IgG has a strong affinity to pG and rat IgG a moderate affinity. Thus, the differential affinity between human IgG and rat IgG to protein G (pG) enables the quantification of goat IgG using rat IgG (see Examples, FIGS. 5 and 8).

The inventors of the present disclosure found the system as described herein may be used to detect agent (analyte/target antibody) in a sample in two different ways. Thus, the system as described herein may be provided in two different settings/embodiments. In one example, the system may be provided as two different systems of either: a displacement assay or a competition assay. In both assays, the quantification of immunoglobulin analyte/target is based on its competitive binding to the Fc binding protein that affects the binding of the fluorescent immunoglobulin. The detectable signal is the change of fluorescent intensity which is dependent on the concentration of the immunoglobulin analyte/target. The difference between the two different systems lie in the preparation of the systems and how the analyte/target is detected. In one example, the system, when provided to the sample as a competition assay, may comprise fluorescing antibody which is not bound (i.e. previously bound) to the Fc binding protein. In another example, the system, when provided to the sample as a displacement assay, may comprise the fluorescing antibody, which is bound to the Fc binding protein. In one example, the system as described herein uses Fc binding protein (such as protein G) coated with nanoparticle as the major sensing element for IgG detection coupled with FRET (Frster resonance energy transfer) and NSET (nanometal surface energy transfer) principle.

As used herein, the term “FRET” refers to a Frster resonance energy transfer, also known as a fluorescence resonance energy transfer. This transfer describes a mechanism by which the energy from an absorbed photon can be disposed of. In the case of FRET, the energy of the excited molecule (the donor) passes directly and without emission of a photon (non-radiative) to a nearby molecule (the acceptor), thereby exciting the acceptor molecule. The acceptor molecule can then decay into its ground state either by fluorescence or non-radiative channels. As used herein, the term “NSET” refers to nanometal surface energy transfer. This transfer refers to a dipole-surface energy transfer process. NSET has a similar energy transfer nature as FRET, but different from FRET that considers the energy transfer between a donor and an acceptor as a dipole-dipole interaction, NSET considers the acceptor as a metallic surface with free conduction electrons, and the energy transfer occurs because of interaction between the dipole of the donor with the electronic continuum in the metallic surface. Without wishing to be bound by theory, it is believed that both FRET and NSET are the principles behind fluorescence quenching by nanoparticles (such as gold nanoparticle/AuNPs).

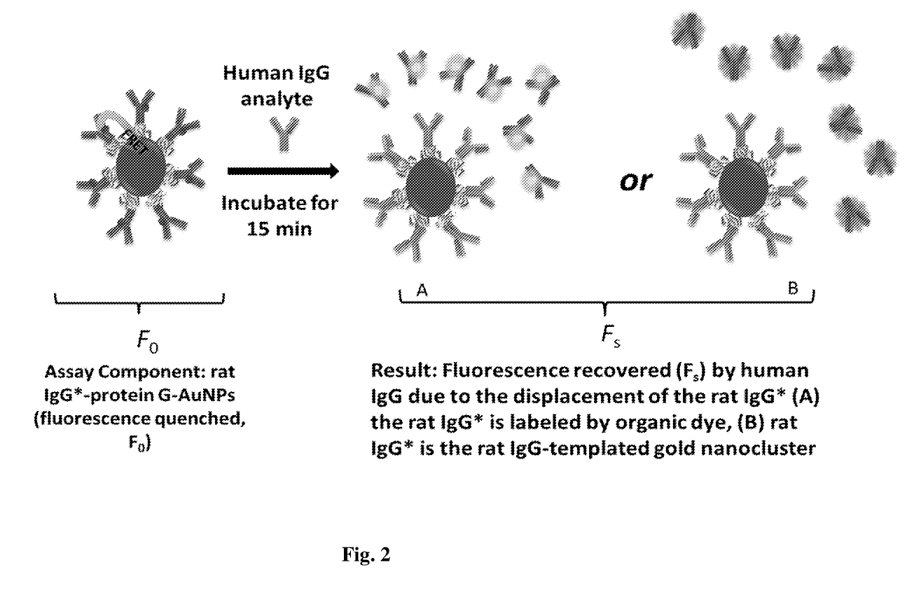

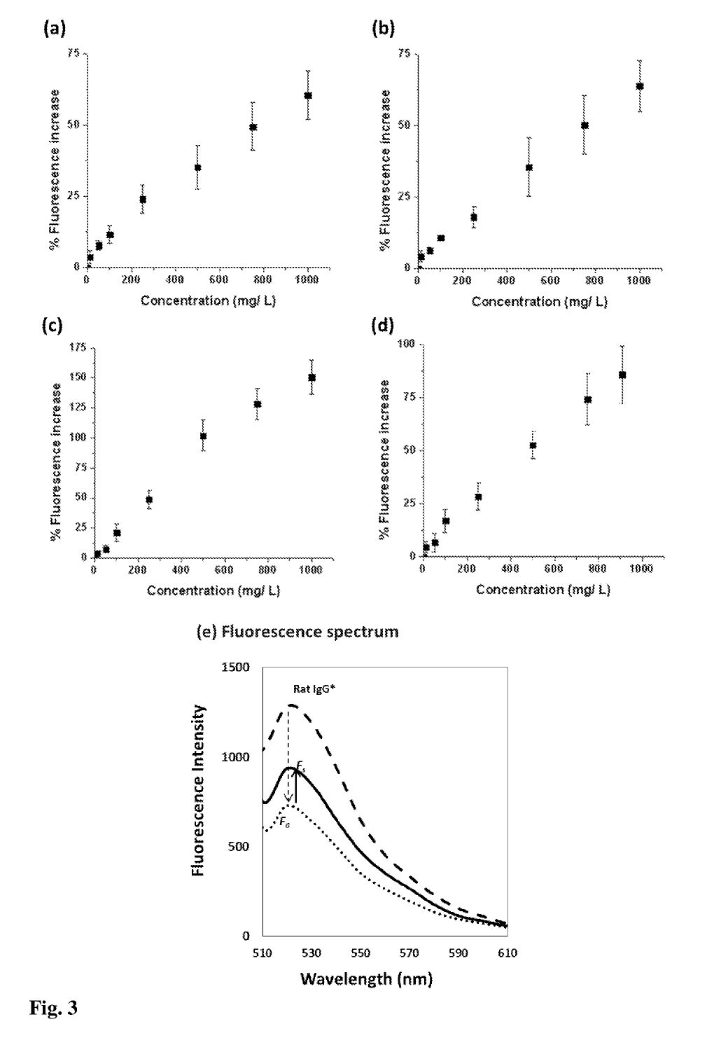

When used in a displacement assay, the system may be used as described in yet another aspect of the present disclosure. In one example, the displacement assay is illustrated by FIG. 2. In this other aspect, the present disclosure provides a method of measuring presence and/or amount of a target antibody in a sample. In one example, the method may comprise the step of (i.e. step a) mixing the sample with the components of the system as described herein. That is, the method as described herein may comprise the composite of the present disclosure/composite made of the components as described herein. In one example, the method further comprise the second step of (i.e. step b) measuring the change in fluorescent intensity observed in the mixture obtained in the first step (i.e. step a) as compared to the total fluorescent intensity of the system before the mixing in the first step (i.e. step a), wherein an increase in fluorescent intensity indicates the presence of target antibody in the sample. Without wishing to be bound by theory, the increase in fluorescent intensity occurs when the quenched fluorescing antibody (which has weaker binding affinity to the Fc binding protein) is released or displaced from the Fc binding protein-nanoparticle complex by the analyte/target antibody. The release of the fluorescing antibody causes the quenched fluorescence to be unquenched by the nanoparticle, which had initially quenched the fluorescence due to proximity. The release of the fluorescing antibody from the Fc-binding protein-nanoparticle complex causes the increase of fluorescence in the sample.

In one example, the fluorescent intensity is calculated using the formula (I):

% Fluorescence increase=(FₛF₀)/F₀×100% (I),

wherein Fₛ is the fluorescence intensity of sample and F₀ is the fluorescence intensity of the system before the mixing in step (a).

In one example, the increase in fluorescent intensity occurs when the target antibody displaces the antibody comprised in the system. Accordingly, in one example, the percentage of fluorescence increases as a function of the titer/amount of the target antibody.

For example, in displacement assay, which is illustrated in Example 1 below, IgG analyte/target, which is a human IgG, is detected based on fluorescence intensity increase, which is induced by the displacement of the fluorescent rat IgG (rat IgG*) from the composite of rat IgG*−Fc binding protein (e.g. protein G/pG) nanoparticles (e.g. gold nanoparticle). In the initial assay component (as exemplified in example 1), the fluorescence intensity of rat IgG* is quenched by nanoparticle because rat IgG* is bound to Fc-binding protein G-coated nanoparticle (e.g. gold nanoparticle) in close proximity. Addition of human IgG, which has a stronger binding strength to Fc-binding protein G than rat IgG, displaces the rat IgG* from the composite. As the rat IgG* is displaced, the emission of rat IgG* is restored and thus increase in fluorescence intensity is observed. The human IgG quantity is restored and thus increase in fluorescence intensity is observed. Thus, in essence, in the displacement assay, antibody part of the component of the system is displaced from the Fc binding protein-coated nanoparticles by target antibody (Ig analyte). The fluorescence intensity may be calculated using the formula (I) above.

When used in a competition assay, the system may be used as described in another aspect of the present disclosure. In one example, the competition assay is illustrated by FIG. 6. In this other aspect, the present disclosure provides a method of measuring presence and/or amount of a target antibody in a sample. The method (competition assay method) may comprise the first step of (i.e. step a) mixing components of a system as described herein and the sample. In one example, the method further comprise the step of measuring the change in fluorescent intensity observed in the mixture obtained in the first step (i.e. step a) as compared to the total fluorescent intensity of the fluorescing antibody before the mixing in the first step (i.e. step a). In one example, a decrease in fluorescent intensity indicates the presence and/or amount (titer) of the target antibody in the sample in an inverse relationship.

In one example, upon reduction in fluorescent intensity, the existing fluorescence detected is referred to as the remaining fluorescent intensity. In one example, the remaining fluorescent intensity is calculated using the formula (II):

% Fluorescence remaining=Fₛ/F₀×100% (II),

wherein Fₛ is the fluorescence intensity of sample and F₀ is the fluorescence intensity of the fluorescing antibody before mixing in the first step (i.e. step a). Thus, in one example, the percentage of remaining fluorescence increases as a function of the titer (amount) of the target antibody.

For example, in a competition assay, which is illustrated in Example 2 below, may be performed by first mixing the fluorescing antibody (e.g. rat IgG*) with sample containing the target/analyte antibody (e.g. human IgG). Then, the Fc-binding protein (e.g. protein G)-nanoparticle (e.g. gold nanoparticle) conjugates are added into the mixture. Depending on the amount of target/analyte antibody (e.g. human IgG) in the sample, inverse amount of fluorescing antibody (e.g. rat IgG*) will bind to Fc-binding protein (e.g. protein G)-nanoparticle (e.g. gold nanoparticle) conjugates, detectable as certain degree of fluorescence decrease from its original intensity (F₀) The remaining fluorescent intensity (Fs) is thus proportionally related to the amount of target antibody (e.g. human IgG), because the binding of target antibody (e.g. human IgG stronger binding strength to protein G than rat IgG*) leaving fluorescing antibody (e.g. rat IgG*) free in solution, thus maintaining its fluorescence intensity (because the fluorescence of the fluorescing antibody will not be quenched by the nanoparticle). If no target antibody is present, more fluorescing antibody would bind to the Fc-binding protein-nanoparticle complex, thus resulting in less fluorescence detected. In contrast, if more target antibody is present, less fluorescing antibody would be able to bind to

Fc-binding protein-nanoparticle complex, thus resulting in more fluorescence detected. Thus, in essence, in the competition assay, antibody part of the component of the system is used to compete with the target antibody (analyte Ig) for binding sites on the Fc binding protein-coated nanoparticles. The remaining fluorescence intensity may be calculated using Formula II above.

In any of the examples of the present disclosure, as used herein, the term “sample” may refer to any samples or analytes containing the agent to be measured for presence and/or amount. In one example, the samples may, for example, include samples derived from or comprising whole blood, serum, plasma, tears, saliva, nasal fluid, sputum, ear fluid, genital fluid, breast fluid, milk, colostrum, placental fluid, amniotic fluid, perspirate, synovial fluid, ascites fluid, cerebrospinal fluid, bile, gastric fluid, aqueous humor, vitreous humor, gastrointestinal fluid, exudate, transudate, pleural fluid, pericardial fluid, semen, upper airway fluid, peritoneal fluid, fluid harvested from a site of an immune response, fluid harvested from a pooled collection site, bronchial lavage, urine, biopsy material, e.g. from all suitable organs, e.g. the lung, the muscle, brain, liver, skin, pancreas, stomach, etc., a nucleated cell sample, a fluid associated with a mucosal surface, hair, or skin. In one example, the sample may be a supernatant of a cell culture system or cell culture medium. In one example, the sample may be ascites of a mammal.

In one example, the target may be an antibody. In one example, the target antibody may be immunoglobulin G (IgG). As would be easily adapted by the person skilled in the art knowledgeable of the various differential affinity of antibodies, the methods as described herein may be used to measure or determine the presence and/or amount of immunoglobulin classes within a sample.

In one example, the methods as described herein may be provided as a high-throughput device. In one example, the high-throughput device may be provided in a microplate format.

As illustrated in the Examples section of the present disclosure, the method as described herein may be performed in a short period of time. In one example, the measuring in the second steps of the methods as described herein (i.e. step (b)) may be performed after incubating the mixture of step (a) for about 10 to 50 minutes, or about 15 to 40 minutes, or about 15 minutes. As used herein, the term “about”, in the context of time to perform step b of the methods as described herein, may refer to +/−5% of the stated value, or +/−4% of the stated value, or +/−3% of the stated value, or +/−2% of the stated value, or +/−1% of the stated value, or +/−0.5% of the stated value. In one example, the method does not require extended incubation of the sample with the system as described herein. Thus, in one example, the method may only require about 15 minutes of incubation period.

In contrast to the methods of detecting analytes known in the art, for example methods such as ELISA (Enzyme-linked immunosorbent assay), the methods/assays as described herein are in a ‘mix-and-measure’ manner that is faster, simpler and cheaper because the methods as described herein avoid multiple incubation steps and may be washing-steps-free.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

### EXAMPLE 1

### Displacement Assay

Assay components in one example of a displacement assay

The component in this assay for quantifying human IgG is the composite of rat IgG*−protein G-gold nanoparticles (AuNPs). Three material elements needed to prepare this composite, includes:

1. Gold nanoparticles (AuNPs) having a diameter of 13 nm to quench fluorescence intensity of proximate fluorophores;

2. Protein G immobilized on gold nanoparticles (AuNPs) for capturing human IgG and rat IgG through the Fc region of the IgG;

3. Fluorescent-labeled rat IgG (rat IgG*) as the IgG to be displaced, which is prepared by either post-conjugation of the rat IgG with organic fluorophore or direct synthesis of intrinsically fluorescent rat IgG (such as rat IgG template gold nanoclusters). The fluorophores are chosen based on the overlap of the emission spectrum with the absorbance spectrum of pG-AuNPs (peak at 530 nm), which acts as the fluorescence quencher.

Preparation of the Assay Components

Firstly, pG-AuNPs conjugates were prepared by incubating 1 mL of 5 nM AuNPs with 40 μL of 500 mg/L pG in a 1.5 mL microtube for 15 mins. Protein G was adsorbed on the surface of AuNPs and the pG-AuNPs conjugates were separated from excess pG by centrifugation at 14,000 rpm for 60 mins. Then, the pG-AuNPs conjugates were reconstituted in 1× Phosphate Buffered Saline (PBS) to a total volume of 100 μL, resulting in 50 nM pG-AuNPs.

Secondly, rat-IgG* are prepared either by post-labeling with an Alexa Fluoro 488 (A488) fluorophore (i.e. fluorescent dye) or templating gold nanocluster formation. The rat IgG-labeled with A488 was prepared using a labeling kit (Invitrogen, Alexa Fluor 488 Protein Labeling Kit, 2006). In this method, 0.5 mL of 2 g/L rat IgG in sodium bicarbonate buffer (pH=8.3) was mixed with A488 dye provided in the kit for 1 hour at room temperature. Then, the rat IgG-labeled with A488 was purified in a column containing purification resin and eluted in PBS. The rat IgG-labeled with A488 concentration was quantified using UV/vis spectrometry.

The intrinsically fluorescent rat IgG-templated gold nanoclusters (rat IgG-AuNCs) was prepared by utilizing the biomineralization properties of IgG protein. In a typical synthesis, 0.25 mL of 15.5 mg/mL rat IgG in aqueous solution was mixed with 0.25 mL of 1 mM HAuC1₄ solution, followed by addition of 7.5 μL of 1 M NaOH. The reaction solution was constantly stirred for approximately 36 hours at room temperature to yield the bright red emitting AuNCs. The as-synthesized AuNCs were collected, and dialyzed to remove unwanted salts. The purified AuNCs were extremely stable without significant fluorescence loss for at least 3 months when stored in a 4° C. fridge.

Finally, the composite of rat IgG*-Protein G-AuNPs was prepared by mixing 50 nM protein G-AuNPs conjugates with 50 mg/L rat IgG-A488 in a 5 to 3 volume ratio. Alternatively, it was prepared by mixing 50 nM protein G-AuNPs conjugates with 0.1 mM rat IgG* template AuNCs (rat IgG* basis) in 1 to 1 volume ratio.

Results

The usage of displacement assay for measurement of three types of humanized monoclonal antibodies (Herceptin, Avastin, and Humira) from Chinese Hamster Ovary (CHO) cell lines for cancer treatment was investigated. The usage of displacement assay to goat IgG using a goat anti-biotin antibody as IgG analyte was also investigated.

A) Displacement Assay Using Rat IgG Labeled With A488 for Quantifying Human IgG

For displacement assay using rat IgG labeled with A488, 10 μL of sample was added into a mixture of 8 μL of the composite and 42 μL of PBS. After 15 mins incubation, the fluorescence intensity (excitation at 470 nm, emission at 520 nm) is measured (Fₛ) by using a microplate reader. The increase in fluorescence (% F increase) is calculated using equation (1) above.

FIG. 3 shows the fluorescence intensity increase upon displacement of rat IgG labeled with A488 by Herceptin, Avastin, and Humira at varying concentration from about 10 to 1000 mg/L. All of the antibodies tested resulted in increase of fluorescence intensity. Based on these results, displacement assay can be used to quantify various therapeutic human IgG.

B) Displacement Assay Using Rat IgG Template AuNCs for Quantifying Human IgG

5 μL of sample was added into a mixture of 10 μL of the composite and 40 μL of PBS. After 15 mins incubation, the fluorescence intensity (excitation at 370 nm, emission at 660 nm) is measured (Fₛ) by using a microplate reader. The increase in fluorescence (% F increase) is calculated using equation (1) above.

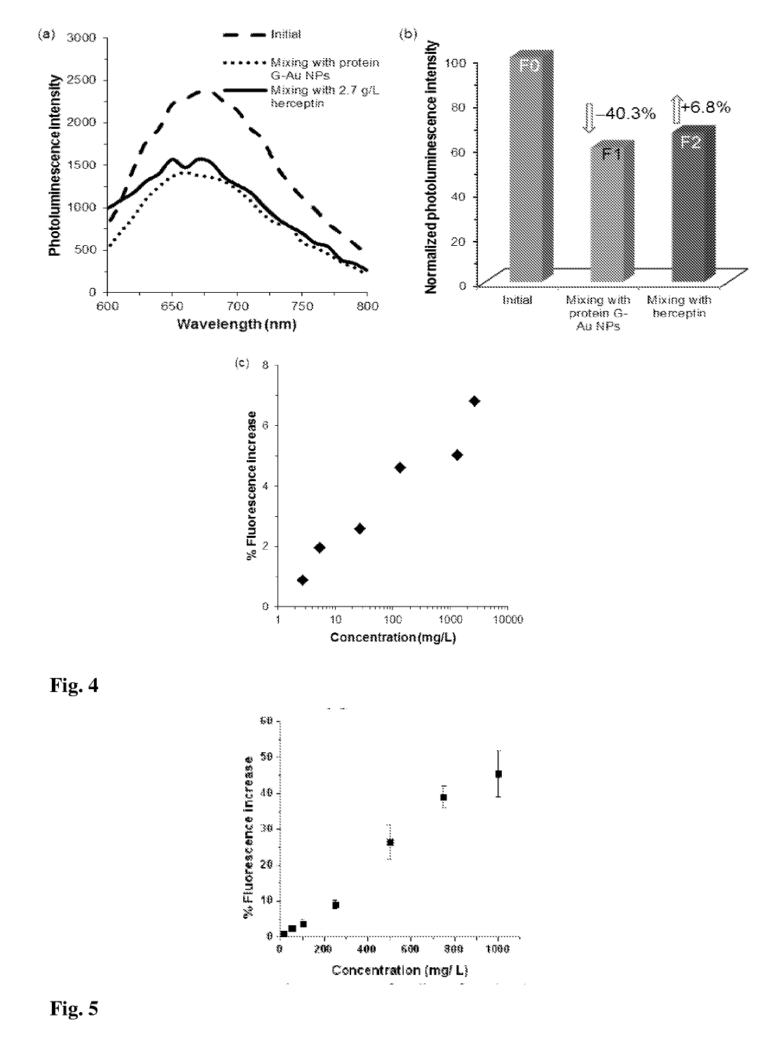

FIG. 4 shows the fluorescence intensity increase upon displacement of rat IgG labeled with AuNCs by Herceptin at varying concentrations of between 2.7 to 2700 mg/L. A linear response correlated to the concentration of sample IgG was observed.

C) Extended Application of Displacement Assay for Quantifying Goat IgG

The inventors of the present application extended the applicability of displacement assay for quantifying IgG from other sources, such as goat IgG (goat anti-biotin antibody). Having goat IgG as the IgG analyte, the Fc binding protein used is pG and the fluorescent IgG (to be displaced) is from rat (rat IgG has a weaker binding affinity to pG than goat IgG).

Using composite of rat IgG* (labeled with A488)-pG-AuNPs and the same assay method used in quantification of human IgG, the study quantify goat anti-biotin in PBS with concentration of 10 to 1000 mg/L.

FIG. 5 shows that the goat anti-biotin tested resulted in fluorescence intensity increase upon displacement of rat IgG labeled with A488 by goat anti-biotin.

### EXAMPLE 2

### Competition Assay

Assay Components in One Example of a Competitive Assay

The components in this assay for quantifying human IgG includes:

1. Rat IgG*;

2. pG-AuNPs conjugate that are in separate entity.

Rat IgG* is chosen as the competitor for IgG analyte to bind to protein G on AuNPs. The rat IgG* and pG-AuNPs conjugates are from the same preparation as in displacement assay above.

Results

The inventors of the present disclosure demonstrated the usage of competition assay for measurement of three types of humanized monoclonal antibodies produced from Chinese Hamster Ovary (CHO) cell lines: Herceptin, Avastin, and Humira. The study also extend the usage of competition assay to IgG from other sources, such as goat IgG (goat anti-biotin antibody).

A) Competition Assay Using Rat IgG Labeled With A488 for Quantifying Human IgG

For competition assay using rat IgG* labeled with A488, 10 μL of sample was mixed with 3 μL of 200 mg/L rat IgG* and 42 μL of 1×PBS and after that 5 μL of 50 nM pG AuNPs conjugate was added into the mixture. After 15 mins incubation, the fluorescence intensity (excitation at 470 nm, emission at 520 nm) is measured by using a microplate reader. The fluorescent intensity of rat IgG* (without IgG analyte and without pG-AuNPs) is defined as F₀. The remaining fluorescence intensity (% Fluorescence remaining) ater incubation with pG-AuNPs is calculated as follows:

% Fluorescence remaining=Fₛ/F₀×100%,

where Fₛ=fluorescence intensity of sample, and F₀=fluorescence intensity of rat IgG* (without IgG anlyte and without protein G-AuNPs).

FIG. 7 shows the remaining fluorescence intensity in competition assay samples containing Herceptin, Avastin, and humira at varying concentration of between 10 to 1000 mg/L. The percentage of remaining fluorescence increases as a function of the antibodies concentration.

B) Extended Application of Competition Assay for Quantifying Goat IgG

The study extend the applicability of competition assay for quantifying IgG from other sources, such as goat IgG (goat anti-biotin antibody) by using rat IgG-labeled with A488 and pG-AuNPs in the same method as quantification of human IgG.

FIG. 8 shows that the percentage of remaining fluorescence intensity increases as a function of goat anti-biotin concentration of between 10 to 1000 mg/L. This result shows the applicability of competition assay as described herein for quantifying IgG from other sources.

1. A system for measuring presence and or amount of a target antibody in a sample comprising:
nanoparticle adapted to quenching fluorescence emission;
an Fc binding protein, wherein the Fc binding protein is immobilized or absorbed on the nanoparticle; and
is fluorescing antibody adapted for binding to the Fc binding protein and having a lower binding affinity to the Fc binding protein than the target antibody.

2. The system of claim 1, wherein the Fc binding protein is not an immunoglobulin that recognize immunoglobulin through antigen binding site.

3. The system of claim 1, wherein the Fc binding protein is selected from the group consisting of Fc receptors, protein G, protein A, and fusion protein G/A.

4. The system of claim 1, wherein the Fc binding protein is immobilized to the nanoparticle by covalent binding of the Fc binding protein with the gold through cysteine residues within the Fc binding protein.

5. The system of claim 1, wherein the Fc binding protein is adapted to differentially binding to the fluorescing antibody and the target antibody.

6. The system of claim 1, wherein the quenching of fluorescence emission occurs when the nanoparticle absorption spectra overlaps with the emission spectrum of fluorescence to be absorbed.

7. The system of claim 1, wherein the nanoparticle is isotropic or anisotropic in dimension.

8. The system of claim 7, wherein the nanoparticle is a spherical nanoparticle or a rod nanoparticle.

9. The system of claim 1, wherein the nanoparticle is about 2 nm to about 80 nm in diameter.

10. The system of claim 1, wherein the nanoparticle is a metal nanoparticle with Plasmonics fluorescence quenching property.

11. The system of claim 10, wherein the nanoparticle is a gold nanoparticle.

12. The system of claim 1, wherein the fluorescing antibody is an antibody, which intrinsically fluoresced, or is labeled with a fluorophore.

13. The system of claim 12, wherein the antibody, which intrinsically fluoresced, is an antibody templated nanocluster.

14. The system of claim 13, wherein the antibody template nanocluster is prepared by mixing the antibody, a nanoparticle precursor solution and a suitable basic solution.

15. The system of claim 1, wherein the antibody is an
immunoglobulin (IgG) having a relatively low affinity to the selected Fc binding relative to the target IgG.

16. The system of claim 1, wherein when provided to the sample, the fluorescing antibody is not bound to the Fc binding protein.

17. The system of claim 1, wherein when provided to the sample, the fluorescing antibody is bound to the Fc binding protein.

18. A method of measuring presence and/or amount of a target antibody in a sample, wherein the method comprises:
a, mixing components of a system and the sample, wherein the system comprises a system for measuring presence and/or amount of a target antibody in sample comprising:
a nanoparticle adapted to quenching fluorescence emission;
an Fc binding protein, wherein the Fc binding protein is immobilized or absorbed on the nanoparticle; and
a fluorescing antibody adapted for binding to the Fc binding protein and having a lower binding affinity to the Fc binding protein than the target antibody; and
b. measuring the change in fluorescent intensity observed in the mixture obtained under a as compared to the total fluorescent intensity of the fluorescing antibody before the mixing under a,
wherein a decrease in fluorescent intensity indicates the presence and/or amount of the target antibody in the sample in an inverse relationship.

19. The method of claim 18, wherein the remaining fluorescent intensity is calculated using the formula (II):
% Fluorescence remaining=Fₛ/FQ×100% (II)
wherein Fₛ is the fluorescence intensity of sample and Fo is the fluorescence intensity of the fluorescing antibody before mixing under a.

20. (canceled)

21. A method of measuring presence and/or amount of a target antibody in a sample, wherein the method comprises:
a. mixing the sample with the components of a system for measuring presence and/or amount of a target antibody in the sample comprising:
a nanoparticle adapted to quenching fluorescence emission;
an Fc binding protein, wherein the Fc binding protein is immobilized or absorbed on the nanoparticle; and
a fluorescing antibody adapted for binding to the Fc binding protein and having a lower binding affinity to the Fc binding protein than the target antibody;
b. measuring the change in fluorescent intensity observed in the mixture obtained under a as compared to the total fluorescent intensity of the system before the mixing under a,
wherein an increase in fluorescent intensity indicates the presence as or amount of target antibody in the sample.

22.-26. (canceled)
